(19) United States
(12) Patent Application Publication
(10) Pub. No.: US 2026/0294679 A1
(43) Pub. Date: Oct. 1, 2026

(54) LIQUID DISPENSING SYSTEMS AND METHODS

(71) Applicant: Bausch + Lomb Ireland Limited, Dublin (IE)

(72) Inventors: Erwin Peter BENIRSCHKE, Dublin (IE); Sylvain George Louis FISCHER, Dublin (IE); Stephen J. PABOOJIAN, Dublin (IE); Grant Timothy Lewis SMETHAM, Dublin (IE); Matthew Daniel CHANDLER, Dublin (IE); David BATEMAN, Dublin (IE); Freddie Charles RIDOUT, Dublin (IE)

(21) Appl. No.: 19/483,128

(22) PCT Filed: May 10, 2024
(86) PCT No.: PCT/EP2024/062975

§ 371 Date: Nov. 10, 2025

Related U.S. Application Data
(60) Provisional application No. 63/501,790, filed on May 12, 2023.

(51) Int. Cl. A61F 9/00; A61J 1/06

(52) U.S. Cl. CPC A61F 9/0008; A61J 1/06

(57) ABSTRACT

A modular handheld fluid dispensing system is disclosed. The system can include a fluid cartridge including an ampoule, a reservoir in fluid communication with the ampoule, an aperture designed to be selectively opened to allow fluid from the ampoule and accommodated in the reservoir to be dispensed therethrough, and an actuator designed to impart vibrations to the fluid cartridge to generate hydrodynamic pressure in the fluid to open the aperture and dispense the fluid; a control cartridge can be removably coupled to the fluid cartridge, the control cartridge including a power source and controller to initiate actuation of the actuator; and a cover pivotally moveable between a closed configuration to prevent fluid from being dispensed through the aperture and an open configuration to permit the power source to be activated.

### FIELD OF THE INVENTION

The present invention generally pertains to devices for dispensing fluid medicines and more particularly pertains to such devices that store and deliver ophthalmic. preservative-free medicines, specifically configured to increase the ease of use, and enhance patient compliance with dosing instructions for the medicine.

### BACKGROUND

There are a number of prescription drugs available for topical application to the eye to treat a variety of ailments, diseases, and conditions. The standard applicator for ophthalmic solutions is the conventional eyedropper. When using an eyedropper, the drug is taken routinely from the same bottle with no control as to the ingress of outside particulate from entering the bottle. When a conventional eyedropper is used, it can require a high level of dexterity and control to properly apply the drop accurately to the eye. Compounding this issue is that if the eye requires medication, it is not operating at normal effectiveness which further decreases the accuracy of the user. This results in ophthalmic medication being misapplied, not applying the intended amount of medication. For example, a user may apply too much medication, i.e., too many drops, or not enough medication due to misapplication.

Ease of dispensing fluid medicines and compliance with dosing instructions are primary concerns with all patients. For example, dispensing bottles such as ophthalmic squeeze dispensers typically require greater actuation force due to a valve mechanism that seals the dispensing nozzle to prevent bacterial ingress and contamination. Such a system requires much higher pressure to operate, and hence much higher squeeze force is required. In addition, prior art dispensing bottles dispense only in an upside-down orientation which require an inconvenient head maneuver and which together with higher actuation force further increases the inconvenience.

Further, the drug is applied through the force of gravity, carrying the drop or droplets into the eye. Then, after the drop is delivered, a portion of the drug is taken back into the container due to the negative pressure within the eyedropper when the eye dropper is released. The portion of drug “sucked” back into the container can introduce microbials and unwanted dust or particulate into the container.

Another concern is shelf-life of ophthalmic solutions. To increase shelf-life of these ophthalmic solutions and to combat any infectious agents, preservatives and other agents are introduced into the ophthalmic solutions. These additives can have various negative side effects experienced by the patient or end-user of the ophthalmic medication. For example, some of the most well used preservatives can cause irritation, dry eye symptoms, effects to tear production and maintenance of the tear film, among others.

As such, there is a current, and pressing, need for a solution that can accurately deliver ophthalmic solution while allowing a preservative free solution to be used.

### SUMMARY

A modular handheld fluid dispensing system is disclosed herein. The system includes a fluid cartridge including an ampoule, a reservoir in fluid communication with the ampoule, an aperture designed to be selectively opened to allow fluid from the ampoule and accommodated in the reservoir to be dispensed therethrough, and an actuator designed to impart vibrations to the fluid cartridge to generate hydrodynamic pressure in the fluid to open the aperture and dispense the fluid. The system includes a control cartridge being removably coupled to the fluid cartridge, the control cartridge including a power source and controller to initiate actuation of the actuator. The system further includes a cover pivotally moveable between a closed configuration to prevent fluid from being dispensed through the aperture and an open configuration to permit the power source to be activated.

In some embodiments, the ampoule can be threadedly received in the reservoir. The system can further include a vent tube designed to allow atmospheric pressure to enter the ampoule. A filter can be arranged in the vent tube to prevent foreign contaminants from entering the ampoule.

In some embodiments, the fluid cartridge can further include a membrane arranged opposite the aperture. A pin portion of the membrane can be received within the aperture to prevent fluid from exiting the aperture. The fluid cartridge can further include electrical contacts to electrically connect the fluid cartridge to the control cartridge. The electrical contacts can include contacts to provide identification information about the ampoule to the control cartridge. The fluid cartridge can further include a latch designed to lock the fluid cartridge relative to the control cartridge. The controller can include a printed circuit board configured to send a control signal to the actuator to dispense the fluid. The printed circuit board can be configured to control a number of doses that are ejected by the actuator. The printed circuit board can be configured to provide a signal to a user indicating a number of doses remaining in the ampoule.

In some embodiments, the cover can be pivotally connected to the fluid cartridge such that the cover can pivot from a closed configuration covering the aperture to the open configuration. The cover can be designed to have a length extending from a pivot. point to an edge, such that when the edge is placed against a cheek of a user, the system will be correctly located for dispensing the fluid. The fluid cartridge can include a concave mirror having an orifice extending through a center of the concave mirror, the concave. mirror being disposed proximate to and concentric with the aperture. The cover can be mechanically connected to a switch arm and the switch arm is designed to activate the power source when the cover is in the open configuration and disengage the power source when the cover is in the closed configuration.

In some embodiments, the ampoule can contain lifitegrast ophthalmic solution.

In an embodiment, a method of dispensing a fluid is provided. The method includes, providing a liquid cartridge including an ampoule, a reservoir in fluid communication with the ampoule, an aperture designed to be selectively opened to allow the fluid to be dispensed therethrough, a cover, and an actuator designed to impart vibrations to the liquid cartridge to generate hydrodynamic pressure in the fluid to open the aperture and dispense the fluid. The method includes receiving the liquid cartridge with a control cartridge, the control cartridge including a power source and a controller for sending a control signal to the actuator. The method further includes pivoting the cover from a closed configuration, where the cover prevents fluid from being dispensed through the aperture, to an open configuration to activate the power source; and activating the actuator to dispense the fluid with the controller.

In some embodiments, the method can further include opening the cover from the closed configuration to the open configuration and aligning the cover with an anatomical feature of a user to align the aperture with an eye of the user. The method can further include aligning a mirror, disposed about the aperture of the liquid cartridge, with the eye of the user to accurately dispense fluid to the eye. The method can further include removing the liquid cartridge from the control cartridge and inserting a new liquid cartridge into the control cartridge.

In an embodiment, a method of assembling a device for dispensing fluid is provided. The method includes providing an ampoule containing a fluid to be dispensed and a liquid cartridge including a reservoir having an opening with a vent tube extending there through, an aperture designed to be selectively opened to allow the fluid to be dispensed therethrough, a cover, and an actuator designed to impart vibrations to the liquid cartridge to generate hydrodynamic pressure in the fluid to open the aperture and dispense the fluid. The method further includes aligning the vent tube with a neck of the ampoule and connecting the ampoule to the liquid cartridge; and arranging the combined ampoule and liquid cartridge in a pouch.

In some embodiments, the combined ampoule and liquid cartridge can be configured to be received within a control cartridge, the control cartridge including a power source and a controller for sending a control signal to the actuator.

In some embodiments, the ampoule can contain a lifitegrast ophthalmic. solution.

### BRIEF DESCRIPTION OF DRAWINGS

These and other characteristics of the present disclosure will be more fully understood by reference to the following detailed description in conjunction with the attached drawings, in which:

FIGS. 1A and 1B are perspective views of a fluid dispensing device according to an embodiment.

FIGS. 2A and 2B are perspective views of a fluid dispensing device according to an embodiment.

FIG. 3A is an exploded view of a fluid dispensing device according to an embodiment.

FIG. 3B illustrates various views of the device of FIG. 3A.

FIG. 3C is an exploded view of a fluid dispensing device according to an embodiment.

FIG. 3D-3I illustrate alternative devices according to various embodiments.

FIG. 4 is a perspective view of a drug cartridge according to an embodiment.

FIG. 5 is a side view of the drug cartridge of FIG. 4.

FIG. 6 is a cross-sectional view of the drug cartridge of FIG. 5.

FIGS. 7A and 7B are cross-sectional view of a fluid dispensing device according to an embodiment.

FIGS. 8A and 8B are cross-sectional views of a drug cartridge according to an embodiment.

FIGS. 9A-9C illustrate an operation of the switch arm.

FIGS. 9D-9F illustrate an alternative switch arm according to an embodiment.

FIG. 10A illustrates a method of filling and packaging a drug cartridge.

FIGS. 10B-10D illustrate tamper resistance features according to an embodiment.

FIG. 11 illustrates the methods of use of a fluid dispensing device according to an embodiment.

FIG. 12 illustrates the front, rear, top, bottom, left, right, and perspective views of a drug cartridge according to an embodiment.

FIG. 13 illustrates the front, rear, top, bottom, left, right, and perspective views of a control cartridge according to an embodiment.

FIG. 14 illustrates the front, rear, top, bottom, left, right, and perspective views of a drug dispensing device according to an embodiment.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure relates to handheld devices for dispensing liquids to a desired location, e.g., an eye of a patient. For example, the instant disclosure relates to reusable liquid dispensers, for example, handheld ophthalmic therapeutic dispensers, that provide for reusable and refillable assemblies that can be used without concern that the patient/user will cause the device to malfunction. For example, the instant disclosure provides for a modular device that is easily and safely refillable and easy for a user to replace medication for prolonged use without additional waste. In addition, or alternatively, the instant disclosure provides for a repeatable alignment technique using hardware present on the device to provide an intuitive alignment method for application of ocular medications.

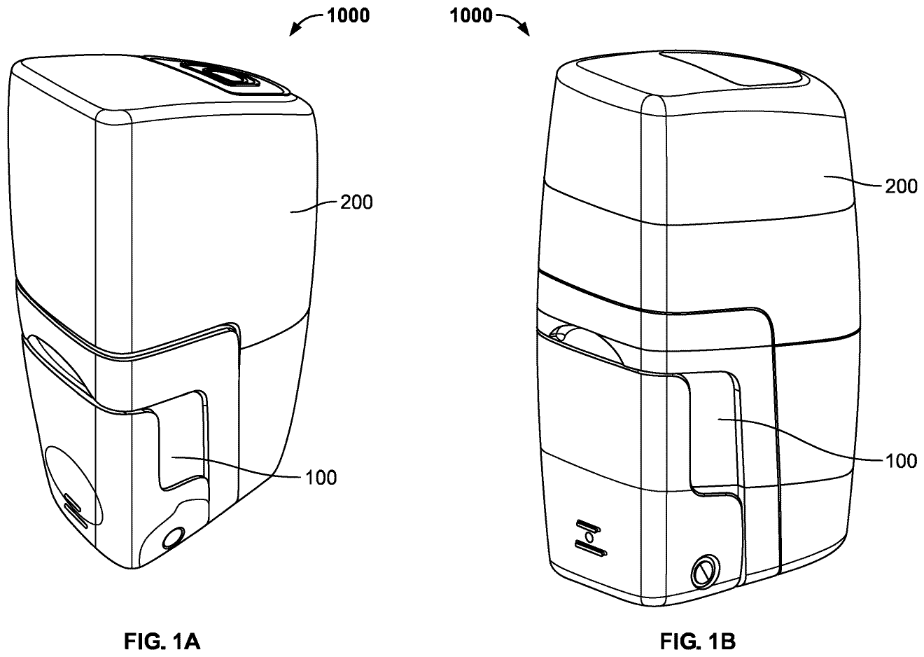

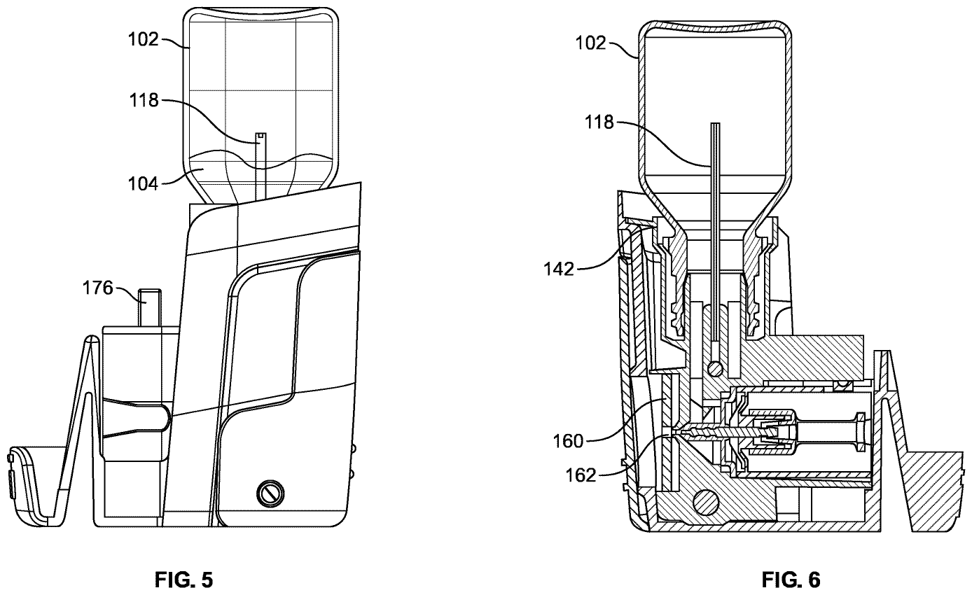

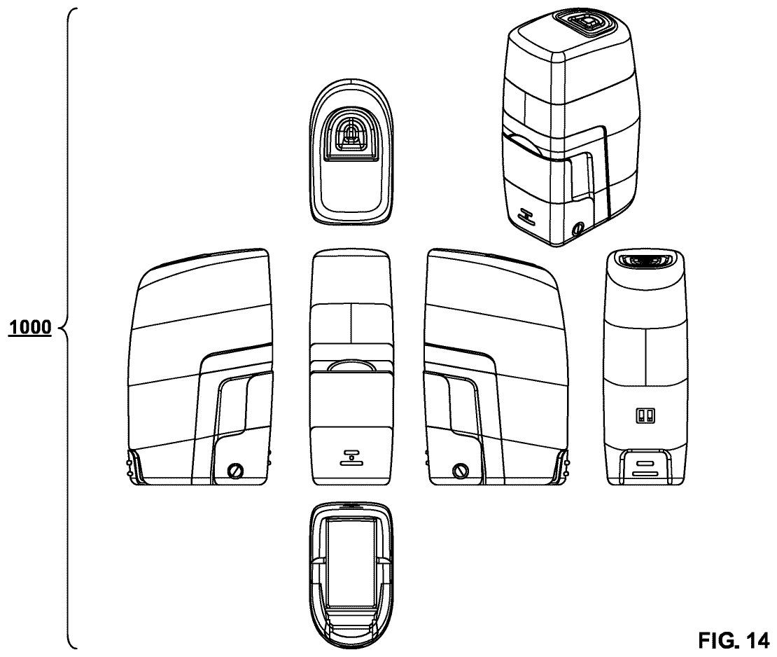

FIGS. 1A-14, wherein like parts are designated by like reference numerals throughout, illustrate an example embodiment or embodiments of the device of dispensing liquid to an eye, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

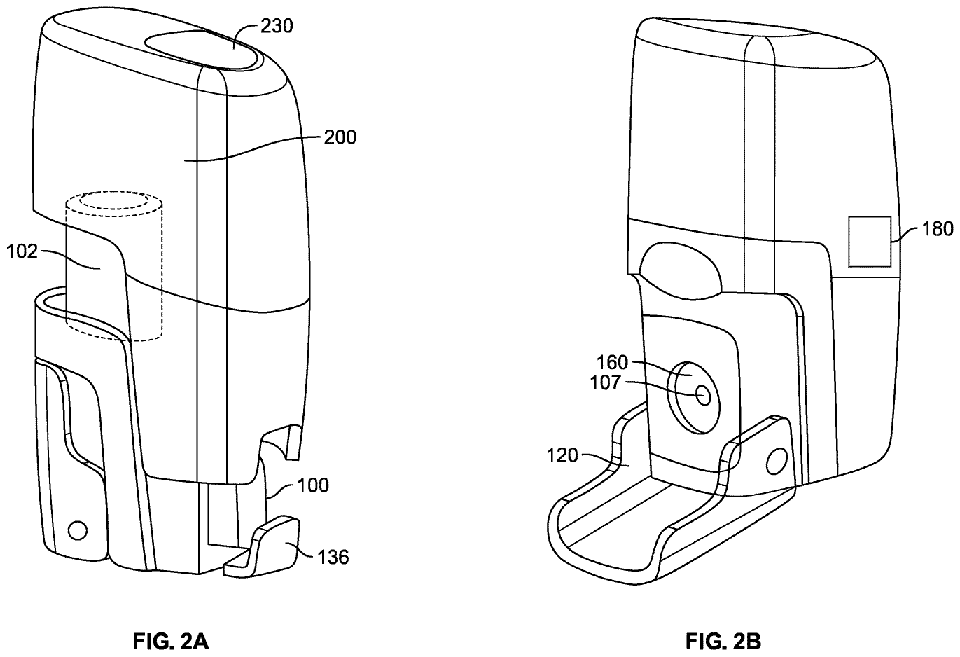

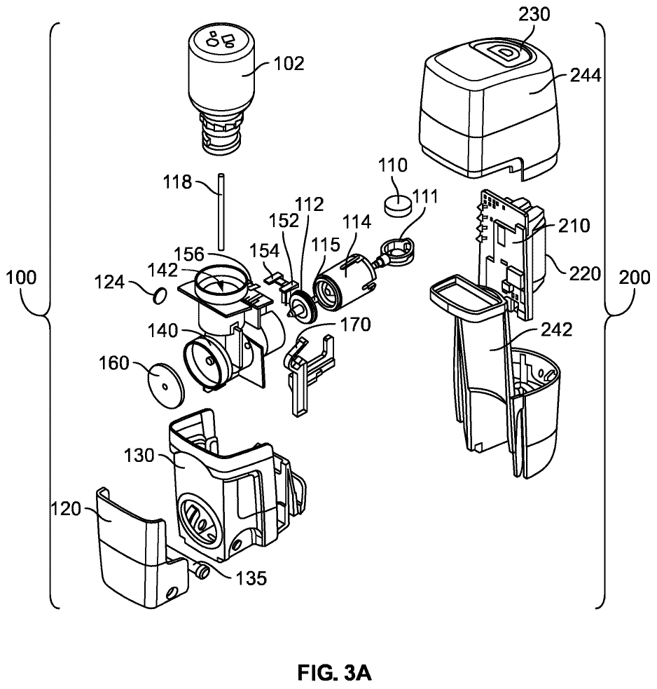

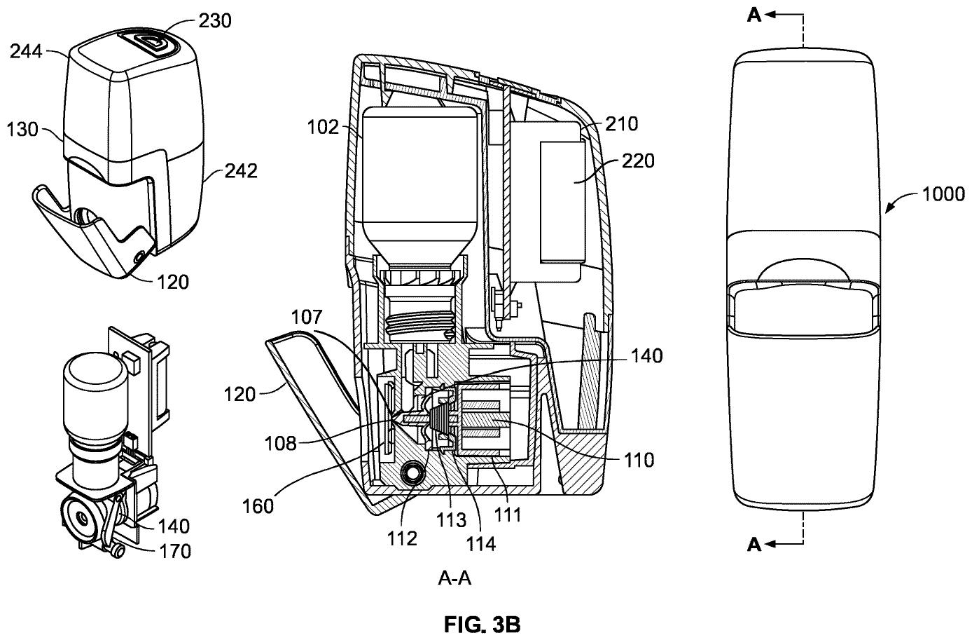

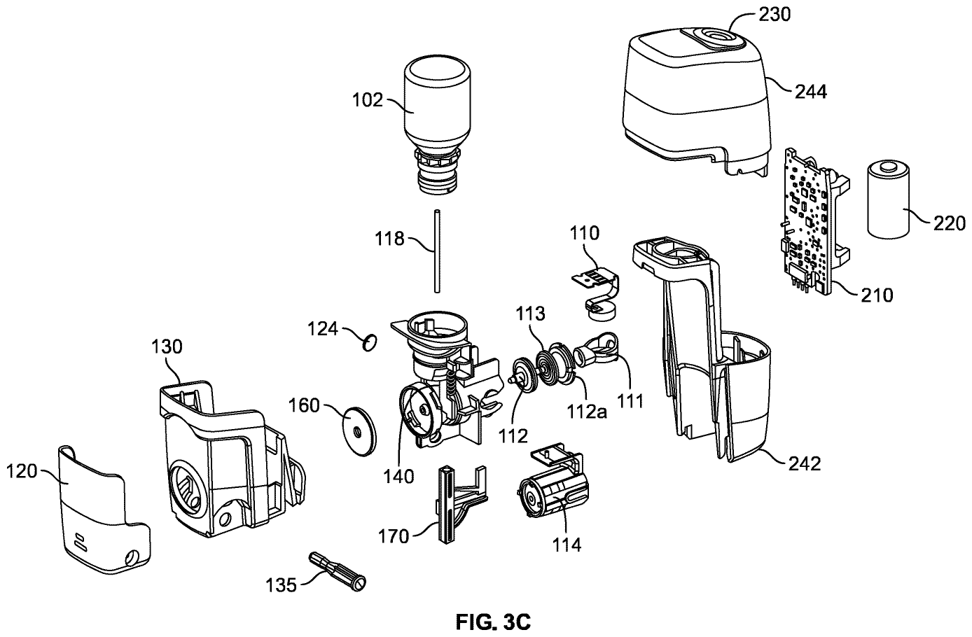

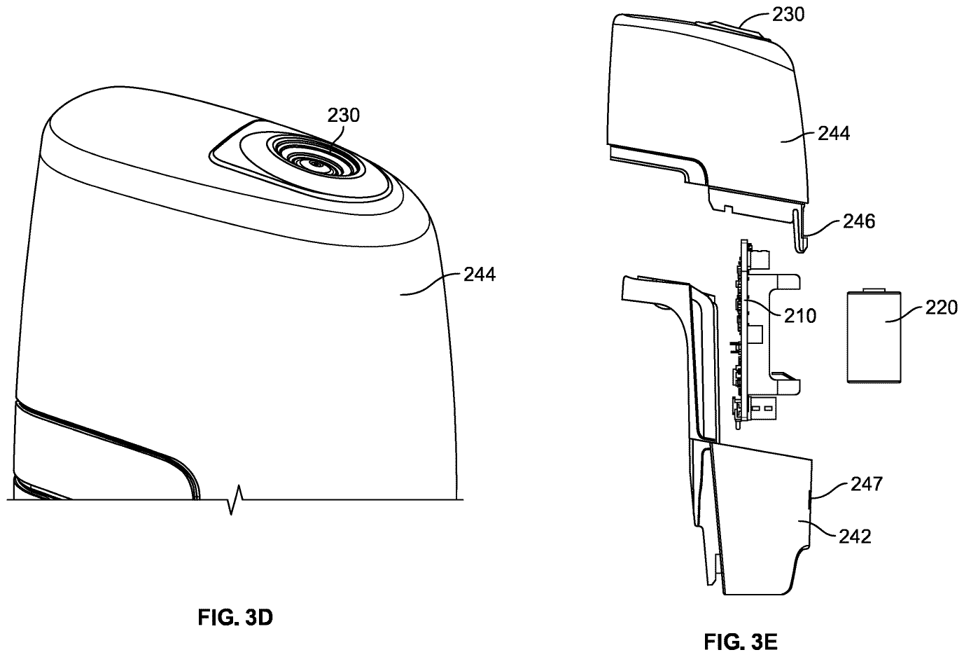

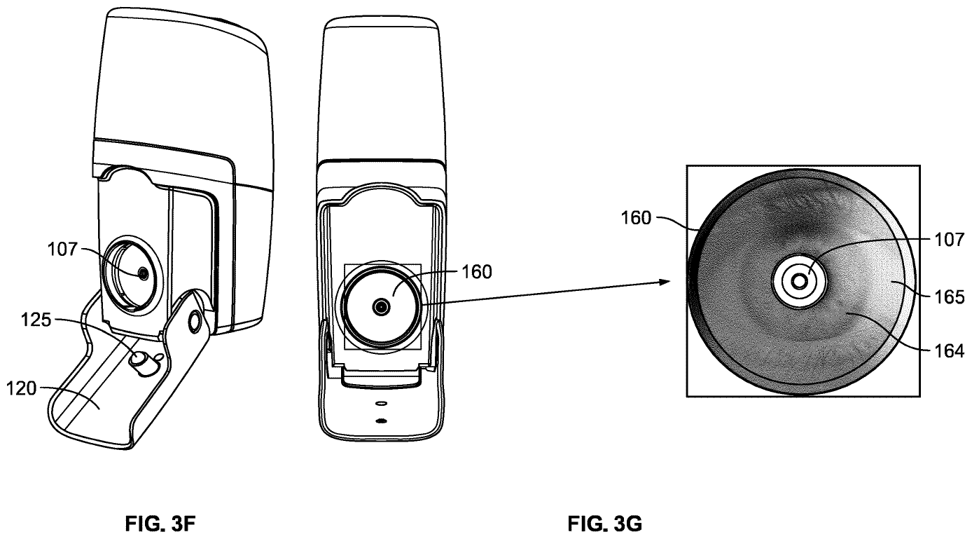

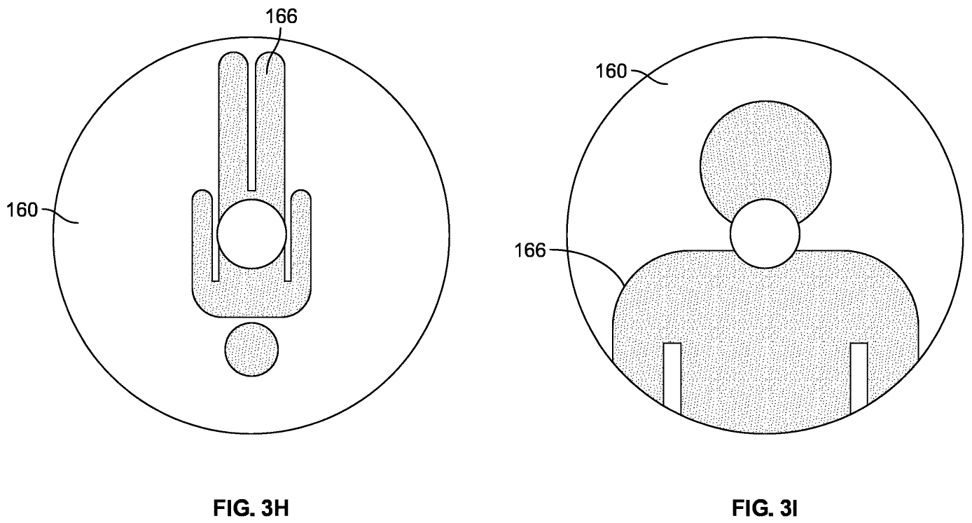

Referring to FIGS. 1A and 1B, the instant device 1000, in accordance with one embodiment of the present disclosure, can be formed from two discrete assemblies, each of which have discrete functionalities and benefits to the overall device. In general, the device 1000 can include a drug cartridge 100 and a control cartridge 200. As shown in FIGS. 2A, 2B, 3A, and 3B, the drug cartridge 100 can include an ampoule 102, containing a liquid 104 to be dispensed, an actuator 110, and a cover 120. The control cartridge 200 can, in general, include a controller 210, a power source 220, and an actuator button 230. When the drug cartridge 100 is removably coupled to, or otherwise engaged with, the control cartridge 200, the device 1000 can be properly aligned with a user's eye and a liquid. 104, which can be an ophthalmic medication, e.g., Xiidra® (lifitegrast ophthalmic solution), can be dispensed to the user's eye. Advantageously, the drug cartridge 100 may contain those portions of the assembly which can be recycled more readily, re-sterilized or irradiated, and refilled. The control cartridge 200, in contrast, may contain portions which are more problematic to recycle, e.g., power source 220 such as batteries, and therefore there is a benefit in being able to continue using the control cartridge 200 after the drug cartridge 100 has been emptied of medication, or liquid 104. Additionally, or alternatively, the instant disclosure provides for a device 1000 where the drug cartridge 100 can easily be replaced while preventing, or minimizing, user error.

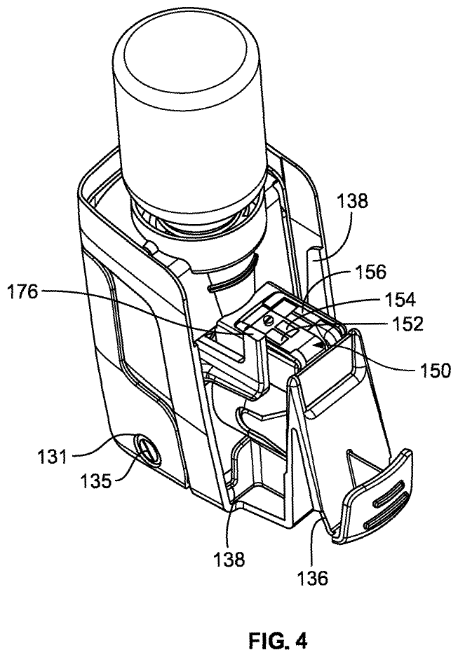

Turning to FIGS. 4-8B, the drug cartridge 100 can include a number of components all contained within a drug cartridge housing 130. For example, the drug cartridge 100 can include an ampoule 102 for containing the liquid 104 to be dispensed, e.g., Xiidra® (lifitegrast ophthalmic solution), to the ocular surface, or a site of interest. The drug cartridge 100 can also include a chamber 140, or fluid reservoir, which can be connected to, and can be in fluid communication with, the ampoule 102 by a channel or fluid pathway 116. The chamber 140 can be defined, generally, as a cavity arranged between an aperture 107 and a membrane 112, where the membrane 112 can act upon fluid 104 within the chamber 140. For example, the membrane 112 can form one of the walls of the chamber 140. In some embodiments, the ampoule 102 can be threaded into an opening 142 of the chamber 140. The opening 142 of the chamber 140 can provide a connection point between the chamber 140 and the ampoule 102, and the opening 142 can function as a pathway for fluid to move from the ampoule 102 to the chamber 140. For example, the neck, or outlet, of the ampoule 102 can have an outer surface having at least one thread and the opening 142 can have an interior surface having a corresponding thread. In an embodiment, liquid 104 can be drawn, e.g., by gravity, through the fluid pathway 116 into the chamber 140 for dispensing. In some embodiments, the fluid pathway 116 can be substantially perpendicular to a fluid ejection path through aperture 107. In some embodiments, the dispensed liquid, e.g., Xiidra® (lifitegrast ophthalmic solution), can have a volume of about 5 μl to about 30 μl. In some embodiments, the dispensed liquid, e.g., Xiidra® (lifitegrast ophthalmic solution), can have a volume of about 12 μl. In other embodiments, the dispensed liquid, e.g., Xiidra® (lifitegrast ophthalmic solution), can be 20 μl or greater such that the average volume of dispensed liquid can be about 28 μl.

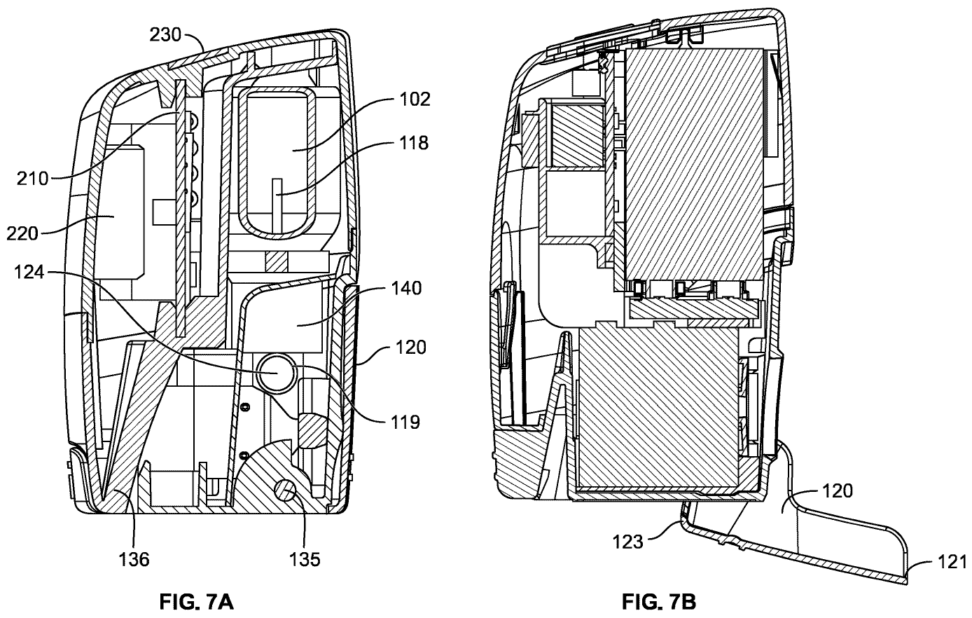

To prevent creation of a pressure vacuum within the ampoule 102, as fluid flows from ampoule 102 to chamber 140, a vent tube 118 can be included in the drug cartridge 100 which can equalize the pressure in the ampoule 102 through the introduction of air from the vent 118. In an embodiment, the vent 118 can be connected to atmospheric pressure at vent inlet 119. The vent inlet 119 can, in some embodiments, extend through the chamber 140 housing but not through the outer housing, or bottom cover 242, as seen in FIG. 7A. To prevent, or minimize, the introduction of most, if not all, contaminants or bacteria, a filter 124 can be included in vent inlet 119. The filter 124 can include any or a combination of filters known in the art for filtering microbes, or other unwanted particulates, from the ambient air.

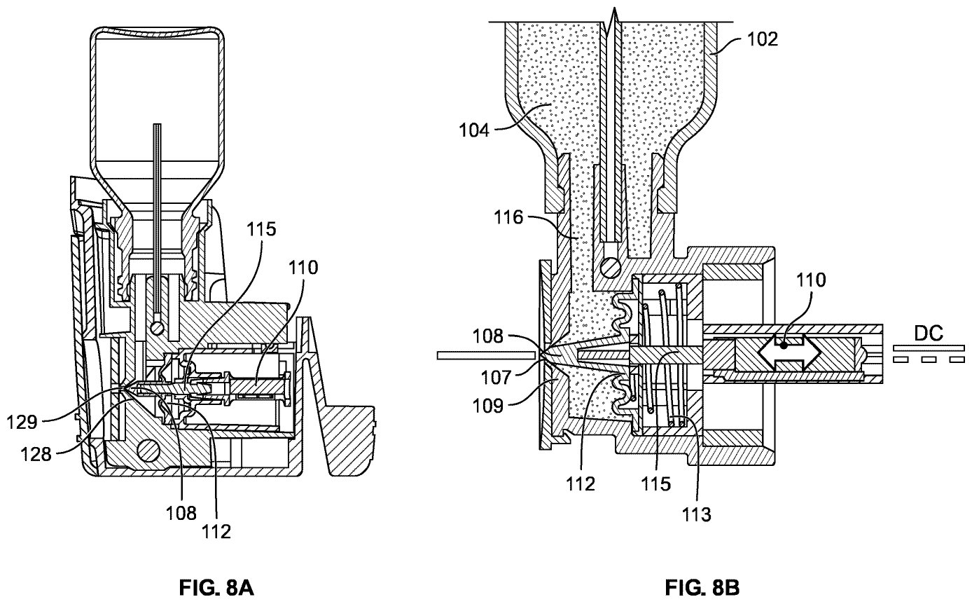

In some embodiments, to prevent ingress of contaminants into the handheld device 1000, the drug cartridge 100 can be provided with a fluid-tight or sealed engagement between an aperture plate, or plate 109, and a member, or pin 108. In some embodiments, the sealed engagement between the aperture plate 109 and the pin 108 can, alternatively, be any sealed engagement connections as disclosed in co-pending U.S. application Ser. No. 17/127,584, filed Dec. 18, 2020, entitled “Vented Multi-dose Ocular Fluid Delivery System,” hereby incorporated by reference in its entirety herein. The sealed engagement of the aperture plate 109 and the pin 108 can provide a selectively closed seal at an outlet, or aperture 107 of the chamber 140. The sealed engagement created by the plate 109 and pin 108 of chamber 140 can be akin to a check valve. As seen in FIGS. 8A and 8B, the plate 109 can include the aperture 107 for dispensing liquid 104 therethrough. The sealed engagement between the pin 108 and the aperture 107 can create a hermetic fluid tight seal. However, the sealed engagement between the pin 108 and aperture 107 can be opened, or disengaged, when a hydrodynamic excitation of the liquid 104 in the chamber 140 reaches a threshold value to open the aperture 107. In one embodiment, the pin 108 can engage the membrane 112, or diaphragm, such that movement of the membrane 112, proximally and distally relative to the aperture 107, can pull the pin 108 at least partially out of contact with the aperture 107. In some embodiments, the pin 108 and the membrane 112 can be a unitary piece, for example formed as one piece. In some embodiments, the membrane 112, or diaphragm can, alternatively, be any membrane 112, or diaphragm and associated arrangements, as disclosed in co-pending U.S. application Ser. No. 17/127,584, filed Dec. 18, 2020, entitled “Vented Multi-dose Ocular Fluid Delivery System,” hereby incorporated by reference in its entirety herein. In some embodiments, the membrane 112 can be retained within a membrane ring 112a, which is in turn received within the chamber 140. When the pin 108 and aperture 107 are disengaged, the liquid 104 can be dispensed through the aperture 107. If the pressure is below the threshold value and the membrane 112 is not actuated, the liquid 104 in chamber 140 can be sealed within the chamber 140; in some cases, the liquid 104 can travel through the pathway 116 up to the ampoule 102. In some embodiments, the aperture 107 can be disengaged from the pin 108 when the pressure within the chamber 140 exceeds about 0.011 MPa to about 0.1 MPa or about 0.014 MPa to about 0.04 MPa. In some embodiments, an axis through the length of the pin 108 is axially aligned with the aperture. In some embodiments, the plate 109 can be made of a flexible elastomer for the sealed engagement. In an embodiment the elastomer can be silicone rubber. Other elastomers which can have a Youngs modulus of elasticity of about 0.1 to about 1.2 GPa can also be used. In some embodiments, the plate 109 can include more than one aperture 107. In other embodiments, the seal between pin 108 and aperture 107 can be any seal.

As can be seen in FIGS. 8A and 8B, the aperture 107, in an embodiment, can be conical and can extend through the plate 109. Alternatively, the aperture 107 can extend through a side wall of the chamber 140. The conical aperture 107 can extend from a larger inlet opening 128 in fluid communication with the chamber 140 and small exit opening 129 through which the liquid 104 can be dispensed. The conical shape can allow the pin 108 to engage with the aperture 107 and allow a more controlled dispensing of liquid 104 through the aperture 107 as the aperture 107 is disengaged from the pin 108 due to the hydrodynamic pressure. Alternatively, in some embodiments, the aperture 107 can have a tubular, tapered, or other shape. The decreasing diameter of the fluid exit pathway in the aperture 107 can impart an increase in fluid velocity as the fluid travels along the exit pathway and out through the aperture 107.

In some embodiments, the aperture 107 can include a mirror 160 arranged on an outer face of the housing 130. The mirror 160 can, advantageously, be used to allow for a user to self-align the device with their eye by seeing their eye in the mirror. For example, the mirror 160 can be circular with a hole 162 arranged at the center. The hole 162 can be approximately as large as the aperture 107. In some embodiments, the mirror 160 can be concave, as viewed from the front of the device 1000. The mirror 160 can be disposed about the aperture such that the hole 162 is concentric with the aperture 107. The mirror 160 can, in some embodiments, be disposed downstream from the aperture 107 and have the hole 162 through which the fluid ejected from the aperture 107 can be directed. In some embodiments, the mirror 160 can be a chrome-3 plated mirror to enhance reflectivity. The mirror can, advantageously, provide a user with the ability to view their pupil centered with a portion of the mirror, e.g., with the hole 162, to provide instant feedback to the user that the device is a correct distance away and aligned with the user's eye for application of the medication. The mirror 160 can, in some embodiments, allow a user to self-align the device by moving the housing 130 relative to the eye until a blurry image within the mirror 160 transitions into an in-focus image within the mirror, indicating alignment of the housing with the eye. In some embodiments, the mirror 160, alternatively, can be any mirror 160, or visual alignment system, for example as disclosed in U.S. Pat. No. 11,278,448, filed May 22, 2019, entitled “Fluid Delivery Alignment System,” hereby incorporated by reference in its entirety herein. It will be appreciated that any reflective. surface, or visual alignment system, can be used in place of mirror 160.

In some embodiments, as shown in FIG. 3G, the mirror 160 can surround an aperture 107 having a dark color, e.g. black, to make it easier to distinguish the iris 164 and sclera 165 of the eye and align the eye to receive the center of the spray. In some cases, the entire chamber 140 can be formed out of a dark color high density polyethylene. In some embodiments, the mirror 160 can be a concave mirror formed from, e.g., plastic as an in-mold chrome plating or post mold chrome 3 plating to provide a bio-compatible surface. The concave mirror can, in some embodiments, provide for a focal distance that is optimized such that the image 166 will be inverted, as seen in FIG. 3H, until the user has located the device 1000 within a desired distance away where the image 166 will flip such that it is right side up, as seen in FIG. 3I. The distance between the user and the mirror 160 where the image 166 is inverted can be greater than about 25 mm in some embodiments.

The pin 108 that is in sealed engagement with the aperture 107 can be formed as part of the membrane 112. In the illustrated embodiment, the pin 108 can be generally tubular and have a distal conical end designed to be received in the aperture 107. Alternatively, in some embodiments, the pin 108 can have a spherical tip designed to be received by the aperture 107. In a further alternative embodiment, the pin 108 can include any complementary, or non-complementary geometric tip can be used. In some embodiments, the pin 108 can include an antibacterial coating or made of an antibacterial material.

In some embodiments, to hydrodynamically excite the liquid 104 in the chamber 140, the membrane 112 can be acted upon, or actuated, by an actuator 110 to create hydrodynamic fluid momentum within the liquid 104. The rapid oscillations, or movement, of the membrane 112 can create fluid momentum in the liquid 104 which can increase the hydrodynamic pressure of the liquid 104 within chamber 140. The vibrations or oscillations imparted to the membrane 112 can create cycles of hydrodynamic pulses which can create momentum and pressure in the liquid 104, thereby causing the aperture 107 to disengage from the pin 108 as the liquid 104 pushes against the aperture 107. In. some embodiments, the actuator 110 can oscillate in a range of about 100 Hz to 200 Hz. For example, the actuator 110 can oscillate at approximately 140 Hz. In some embodiments the pulse displacement of membrane 112 can be about 100 microns to about 250 microns and the pulse can occur within about 100 ms-200 ms. The aperture 107 can be re-sealed by the pin 108 when the fluid pressure is reduced. Alternatively, in some embodiments, the membrane 112 can be a rigid wall, semi-rigid wall, to hydrodynamically excite the liquid 104.

The actuator 110 can include any actuator that is configured to output an oscillating linear motion to pin 108. In some embodiments, the oscillating linear motion can be axially aligned with the aperture 107. In an embodiment, the actuator 110 can be a coin cell motor which can be housed within actuator housing 111, though it will be appreciated that any actuator can be used that generates an oscillating linear motion. Alternatively, in some embodiments, the actuator 110 can be an electromagnetic transducer including solenoid, as disclosed in U.S. application Ser. No. 16/811,879, filed Mar. 6, 2020, entitled “Multi-Dose Ocular Fluid Delivery System,” hereby incorporated by reference in its entirety herein. The actuator housing 111 can be retained within the housing 130 such that the actuator 110 is fixed relative to the housing 130 to impart actuation to a drive pin 115. The drive pin 115 can, in turn, be retained within the membrane 112. In some embodiments, a spring 113, such as a conical helical spring, can be disposed about the drive. pin 115. The spring 113 can compress as the actuator advances the pin 108 towards the membrane 112 and can provide for a return force to aid in moving the drive pin 115 back to a neutral position in a direction away from the membrane 112. The spring 113, disposed about the drive pin 115, can be retained by a spring housing 114. The spring housing 114 can be generally cylindrical, though other shapes are contemplated. In some embodiments, the spring housing 114 can generally aid in directing the spring 113 to ensure that the spring 113 does not kink or bend. Additionally, or alternatively, in some embodiments the actuator housing 111 can additionally be retained by the spring housing 114.

The actuator 110 can, in some embodiments, be electronically connected to electrical contacts 150. The electrical contacts 150 can provide for an interface between the actuator and pogo-pins on the controller 210. Both electrical power and electrical control signals can be transferred at the connection between the controller 210 and the electrical contacts 150. In some embodiments, the electrical contacts 150 can include a first electrical contact 152 and a second electrical contact 154. The actuator 110 can be hardwired to the electrical contacts 150. Alternatively, a hardwired connection can be used to provide electrical power and a wireless connection can be used to wirelessly send signals to the actuator 110. In some embodiments, the drug cartridge 100 can additionally include a plurality of lights 180 to indicate the number of weeks of doses remaining in the ampoule 102. Alternatively, the plurality of lights 180 can be disposed anywhere on the device 1000.

In some embodiments, in addition to the electrical contacts 150, the drug cartridge 100 can include identification (ID) electrical contact pads 156 which can be in contact with the controller 210. The ID electrical contact pads 156 can provide a unique identification signal to the controller 210. Such an identification signal can provide the controller with information pertaining to the type of medication contained in the ampoule 102, the amount of fluid to be ejected per dosage, the number of doses to be administered for each application, the total number of ejections contained in the ampoule 102, and other pertinent medical information. It should be appreciated that other identification means can be included in place of the electrical contacts 150, for example near field communication (NFC) sensors.

In some embodiments, the above noted structure of the drug cartridge 100 can be contained by a housing 130. The housing 130 can, in some embodiments, be ergonomically designed to be comfortably held in a user's hand and can be formed of any suitable material, such as antimicrobial materials including plastics and metals. The housing 130 can, in some embodiments, include a top opening and a rear opening. The rear opening can extend through to a sprung latch 136. The sprung latch 136 can be an inverted “V” shaped living hinge latch that can be used to retain the control cartridge 200 to the drug cartridge 100. For example, the sprung latch 136 can be compressed when the control cartridge 200 is slid down along ribs 138 of the drug cartridge 100. Once the control cartridge 200 is completely depressed down onto the drug cartridge 100, the sprung latch 136 can expand and lock the control cartridge 200 to the drug cartridge 100.

In some embodiments, the housing 130 can additionally include a cover 120. The cover 120 can provide for a number of different functionalities to the device 1000. The cover 120 can be pivotally connected to the housing 130 via an axel 135, that extend out of either side of the housing 130. The cover 120, in turn, can include two through holes 131 which can receive the axel 135 to allow the cover 120 to pivot from a closed configuration, as seen in FIG. 7A, to an open configuration, as seen in FIG. 7B. In the closed configuration, the housing 130 can include a soft lock (not shown) to retain the cover 120 against the housing. In the open configuration, a distal edge 123 can be disposed against the bottom surface of the drug cartridge 100 and similarly, locked in position with a soft lock (not shown). The cover 120, can include a proximal edge 121, which can be used for an initial alignment of the device with respect to the user's eye. For example, when the cover 120 is in the open configuration, the proximal edge 121 can be arranged against a user's cheek to indicate to the user that the device is at a proper distance from the user's eye, for example approximately 2.5 cm. This alignment technique can be performed independent of any alignment using the mirror 160.

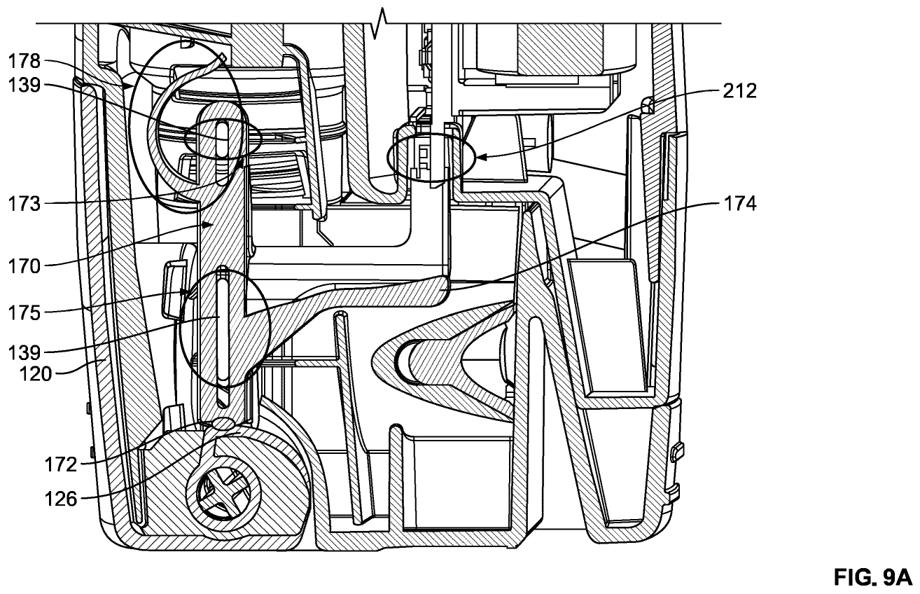

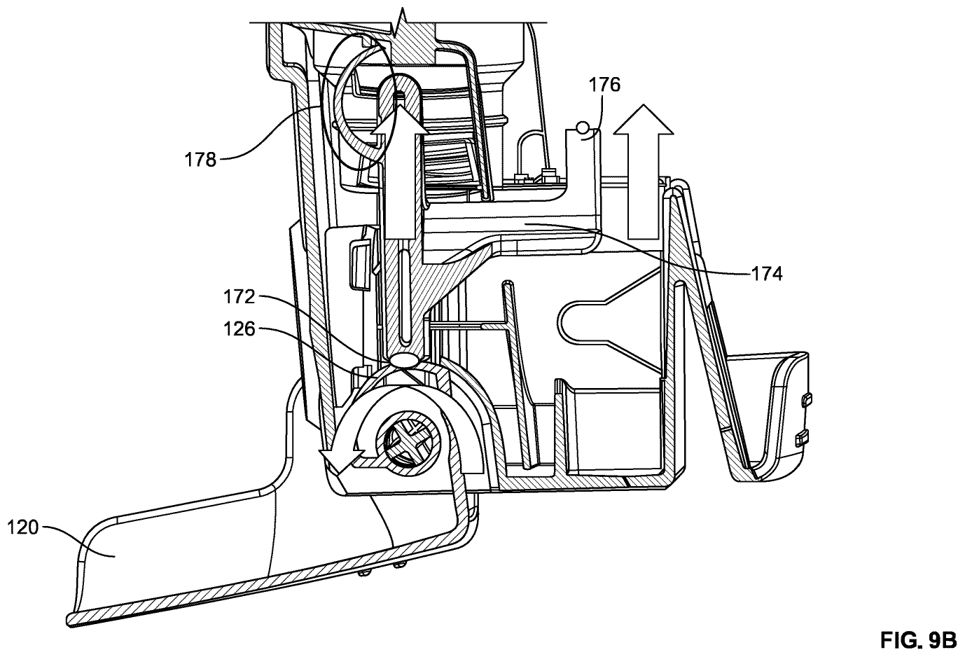

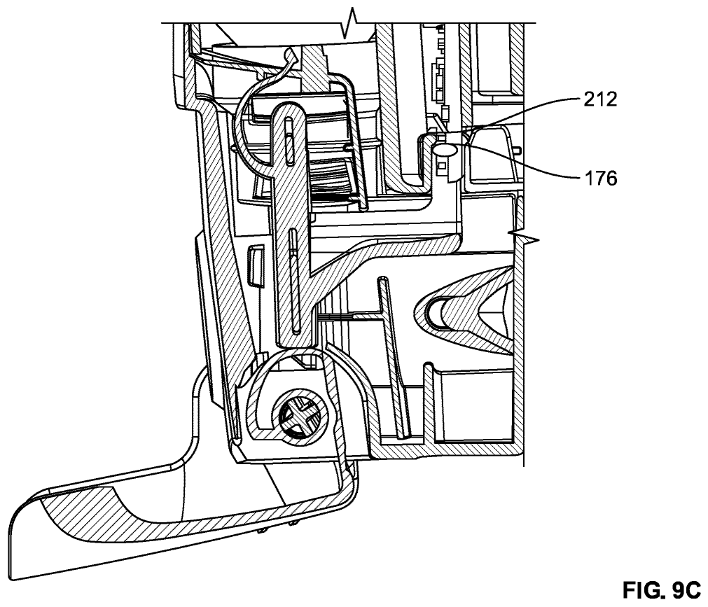

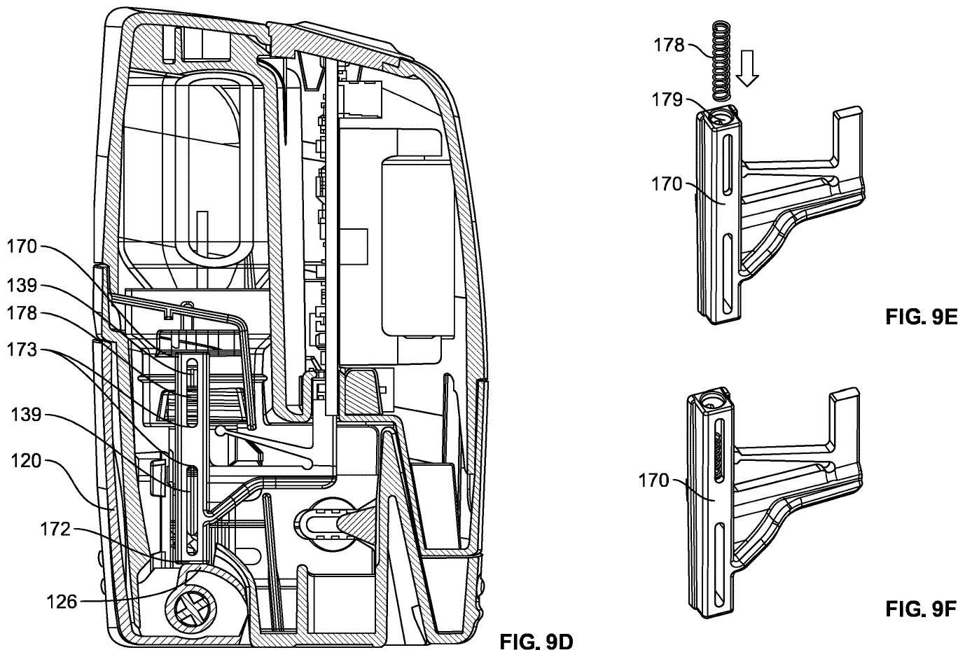

In addition to functioning as a guide for the user, the cover 120 can additionally be used to activate, or power on, the device 1000. For example, as shown in FIGS. 9A-9C, the drug cartridge 100 can include a switch arm 170 which can be actuated by a cam surface 126 of the cover 120. As the cover 120 is pivoted from the closed configuration to the open configuration, the cam surface 126 can push against a lower surface 172 of the switch arm 170 to push the switch arm upward. The switch arm 170 can be constrained to linear movement by a channels 173, 175 that can receive guiding ribs 139 of the housing 130. The switch arm 170 can include a switch lever 174 having a distal switch point 176 that can engage a power switch 212 on the controller 210. When the distal switch point 176 is pushing against the power switch 212, the device 1000 can be in a powered on state. When the cover 120 is rotated back into the closed position, a spring arm 178 arranged on an upper end of the switch arm 170 can push the switch arm 170 downward to disengage the distal switch point 176 from the power switch 212 to power the device 1000 off.

In some embodiments, as shown in FIGS. 9D-F, an alternative switch arm 170 having a separable spring arm 178 is provided. Only those portions of the alternative embodiment that are different will be discussed, for the sake of brevity. In some embodiments, the switch arm 170 can include a linear spring, e.g. a helical spring, 178 that is received within a bore hole 179 in the switch arm. The linear spring 178 can function substantially the same as the integral spring arm 178 as shown in FIGS. 9A-C. In some embodiments, the switch lever 174 can have a more robust construction to provide added strength.

In some embodiments, as shown in FIG. 3F, the cover 120 can include a compliant sealing tip 125 extending from an interior surface of the cover 120. For example, the compliant sealing tip 125 can be received within the aperture 107 to prevent leakage of the liquid from the device 1000 during transit or storage. In some embodiments, the compliant sealing tip 125 can prevent clogging that can be caused in the aperture 107 by exposure of the liquid to air.

Turning to FIG. 3A, the control cartridge 200 can be designed to contain those portions of the assembly that are both costly and can have the largest environmental impact. As such, it is advantageous to reuse the control cartridge 200 as many times as possible before discarding. The control cartridge 200 can include an actuator button 230, power source 220, and a controller 210. The actuator button 230 can be depressed to make contact with a switch on the controller 210. In some embodiments, the actuator button 230 can be a separate part that is pivotally connected to the control cartridge 200 via a pivot. Alternatively, the actuator button 230 can be integral with the control cartridge 200 can connected via a living hinge. In a further alternative, the actuator button 230 can be retained within the control cartridge 200 such that the button translates linearly. In some embodiments, as shown in FIG. 3D, the button 230 can be an overmolded button formed from thermoplastic elastomers, or “soft-touch” materials. Overmolding of the button can prevent dirt, or moisture from entering into the top cover 244. The button 230 can have a tactile profile that can enable a user to find a raised “braille” type protrusion to locate the center of the button by feel alone to actuate the button. When the switch is activated, the controller 210 can send a control signal to the actuator 110 such that the actuator 110 is activated by drawing power from the power source 220. The power source 220 can be a wireless battery. In some embodiments, the power source 220 can be retained within the control cartridge 200 such that a patient, or user, cannot replace the power source 220. Alternatively, the power source 220 can be user replaceable.

The power source 220 can provide energy to all portions of the device 1000 via wired, electrical connections. For example, the power source 220 can provide power to the controller 210 for actuation of the device 1000. Electrical power from the power source 220 can be controlled via the controller 210 and transmitted, via first electrical contact 152, second electrical contact 154 to the actuator 110 to activate the actuator. The controller 210 can be a printed circuit board which can contain control instructions, e.g., a computer program stored on non-transitory computer readable medium, and store data. In some embodiments, the controller 210 can include a transceiver to allow data to be sent to, and from, the controller 210 from an external source. The controller 210 can receive identification data from ID electrical contact pads 156 of the drug cartridge 100 to ensure that the proper dose is dispensed when the device 1000 is actuated. The identification data can include security or authentication data, prescribed dosage per actuator button 230 depression, and number of doses contained within the ampoule 102. In some embodiments, the controller 210 can illuminate the correct number of lights 180 to indicate how many weeks of doses may remain in the ampoule 102. In some embodiments, the controller 210 can additionally record dose history to allow a medical practitioner to review compliance with the medication regimen. As noted above, the controller 210 can, additionally, trigger the actuation of the actuator 110. The controller 210 can be programed to perform other functions including updating, via external connection, some or all of the identification data received from the drug cartridge 100.

The components of the control cartridge 200 can be housed in a two part housing. The two part housing can include a first, or bottom cover 242 that can retain a portion of the controller 210 and power source 220. The bottom cover 242 can additionally be shaped to be complementary to the housing 130 of the drug cartridge 100. In some embodiments, the bottom cover 242 can additionally include retaining features (not shown) to retain the sprung latch 136 of the drug cartridge 100 to hold the device 1000 together once assembled. Further, the two part housing can include a second, or top cover 244. The top cover 244 can retain the upper portion of the controller 210 and power source 220. In some embodiments, the top cover 244 can be retained relative to the bottom cover 242 by mechanical or chemical fasteners. For example, as shown in FIG. 3E, the top cover 244 can include a latch 246 extending from a portion of the top cover 244. The latch 246 can be received into a cut out 247 arranged on a portion of the bottom cover 242 such that when the latch 246 is locked into the cut out 247, the top cover 244 is fixed to the bottom cover 242.

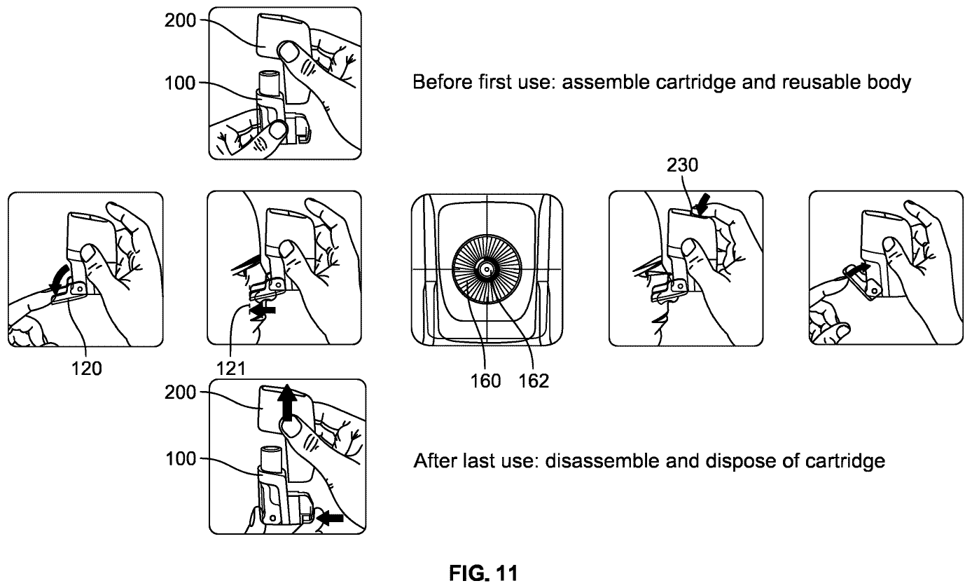

As shown in FIG. 11, the drug cartridge 100 can be inserted within the control cartridge 200 to create the device 1000. As the drug cartridge 100 is inserted within the control cartridge 200, guide ribs 138 on the housing 130 can ensure proper alignment of the control cartridge 200 relative to the drug cartridge 100. In some embodiments, the sprung latch 136 of the drug cartridge 100 can be compressed when the control cartridge 200 is slid down along ribs 138 of the drug cartridge 100. Once the control cartridge 200 is completely depressed down onto the drug cartridge 100, the sprung latch 136 can expand and lock the control cartridge 200 to the drug cartridge 100. To separate the drug cartridge 100 from the control cartridge 200, the sprung latch 136 can be compressed to release the lock and allow the two portions to be separated from one another.

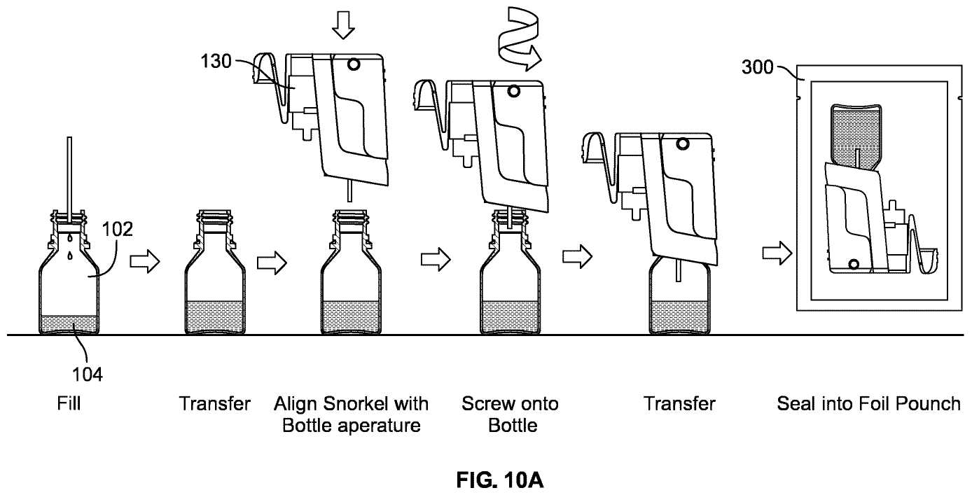

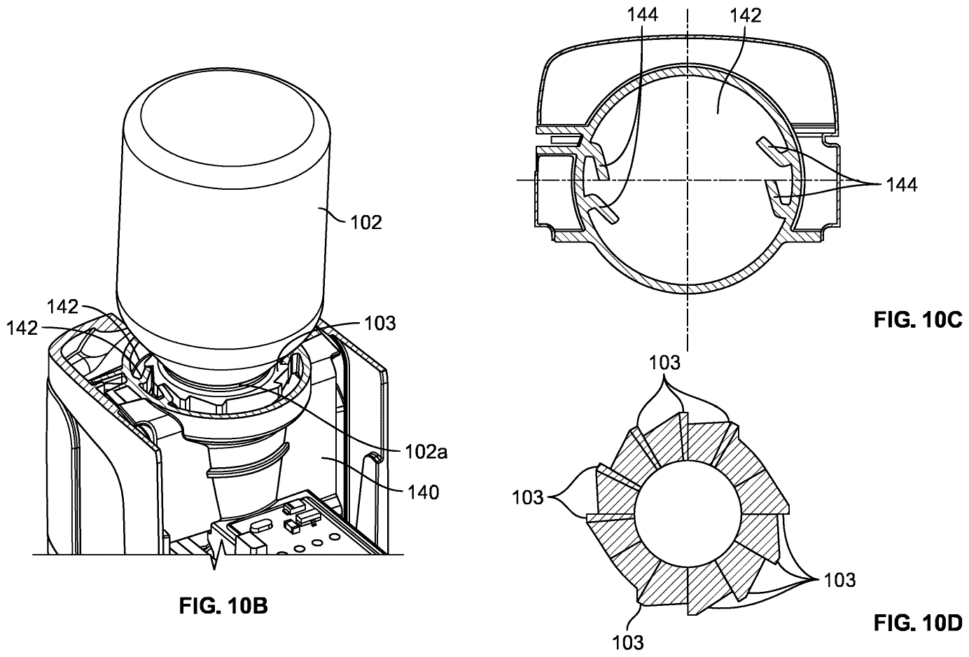

In one method of use, the drug cartridge 100 can be filled and packaged for distribution to users. For example, as shown in FIG. 10A, in a first step, the ampoule 102 can be turned upside down and filled with a desired medication. The filled ampoule 102 can then be transferred to another station, in some embodiments. Once the ampoule 102 is filled to the desired level, the vent 118 of housing 130 can be aligned with the ampoule 102 and the housing 130 can be screwed on to, or otherwise secured to, the ampoule 102. In some embodiments, the drug cartridge 100 can then be labeled and/or inserted into a sealed foil pouch 300. In some cases, the drug cartridge 100 including the ampoule 102 may have been previously used by a user and the entire drug cartridge 100 and ampoule 102 can be sterilized for reuse. This process can be completed any number of times to allow a large number of drug cartridge 100 to be shipped to a single location, e.g., a pharmacy, for distribution to users. In some cases, this method can be completed in a sterile environment to prevent the medication from being contaminated.

In some embodiments, the device 1000 can include tamper resistant features, as shown in FIGS. 10B, 10C, and 10D. The tamper resistant features can include a plurality of teeth 103 extending radially outward of the neck 102a of the ampoule 102. The teeth 103 can be regularly spaced about the neck 102a, or as shown, the teeth 103 can be grouped together, but not equally spaced about the neck 102a. The teeth 103 can interact with a plurality of ribs 144 extending, at least partially, radially and tangentially inward from the chamber opening 142. The teeth 103 can be ramped ridges that allow the teeth to pass over the ribs 144 when the ampoule 102 is being threaded into the opening 142, but prevent the ampoule 102 from being removed from the chamber 140. This can be advantageous because if a user were to independently refill the ampoule 102 without the proper sterilization procedures in place, they can introduce harmful materials. Therefore, the tamper resistant features prevent the user from removing the ampoule 102, or can cause damage to the ribs 144 which can evidence such tampering.

In a method of use, a user can receive the drug cartridge 100. In some cases, the user will already have a control cartridge 200, but if not, they can obtain one from a pharmacy, for example. Before the first use, the user can assemble the device 1000 by inserting the drug cartridge 100 into the control cartridge 200 until they are locked together. In some embodiments, an optional priming step can be performed by depressing the actuator button 230. When the user is ready to use device 1000, the user can pivot the cover 120 to the open configuration, as shown in FIG. 11. Upon pivoting the cover 120 to the open configuration, the switch arm 170 can engage the power switch 212 to power device 1000 on. In some embodiments, the user may check the plurality of lights 180 to ensure. that the device 1000 is still full enough to provide them with a dose.

The user can then use the proximal edge 121 of the cover 120 to initially align the device 1000 with their cheek. This initial alignment can ensure that the device 1000 is at the proper distance for application of the liquid 104 to the eye. Additionally, or alternatively, the user can align the aperture 107 with the pupil of the eye by looking in the mirror 160 to center their pupil within the hole 162. While two alignment steps are described herein, either one, or both, of the alignment steps can be performed. Once the device 1000 is properly aligned with the user's eye, the user can depress the actuator button 230. Upon depression of the actuator button 230, a signal can be sent to the controller 210 which can then initiate actuation of the actuator 110. The actuator 110 can oscillate, or vibrate, the membrane 112 to create sufficient hydrodynamic pressure in the liquid 104 to eject a predefined dose of the liquid 104 to the eye of the user. When the user has completed the dosing regimen, the cover 120 can be pivoted to the closed position which can turn the device off.

In the case where the drug cartridge 100 is empty, the user can disassemble the device 1000 by depressing the sprung latch 136 and lifting the control cartridge 200 away from the drug cartridge 100. If the user has completed their dosing regimen, then the user can discard the entire device 1000. If the user needs to continue with the medication, a new drug cartridge 100 can be inserted into the control cartridge 200 for the next dose. Generally, the drug cartridge 100 can have a sufficient volume medication to last for approximately 30 days. For example, the medication may be delivered to a patient in four distinct doses over the course of a 24 hour period, so the ampoule 102 can accommodate, for example, approximately 120 doses. However, with a larger ampoule 102, the drug cartridge 100 may last longer. While the control cartridge 200 is reusable, it is contemplated that the control cartridge 200 may need to be replaced after, approximately, 90 days. The life of the control cartridge 200 may be a function of the number of doses per day, the size of the batteries, etc. In some embodiments, the device may include a battery that can last longer than 90 days or can be otherwise rechargeable, via a wired or wireless connection.

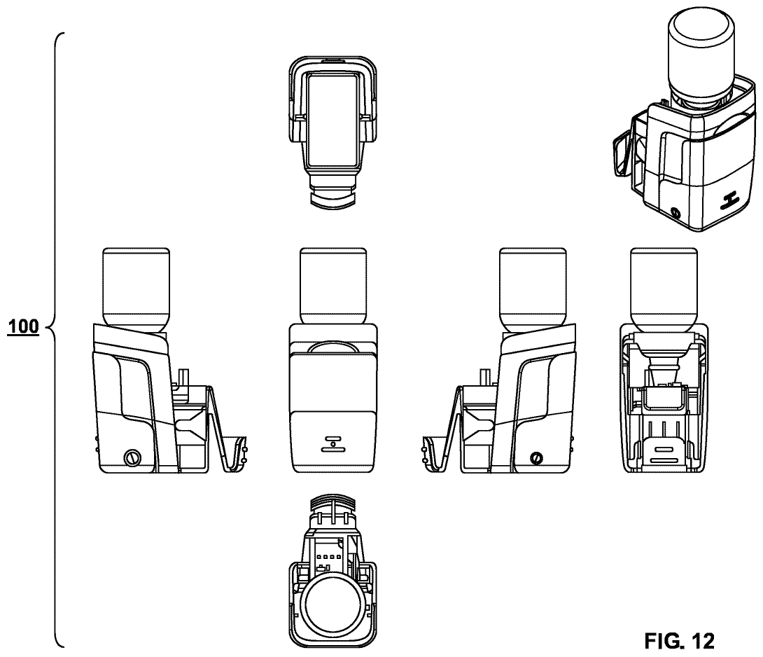

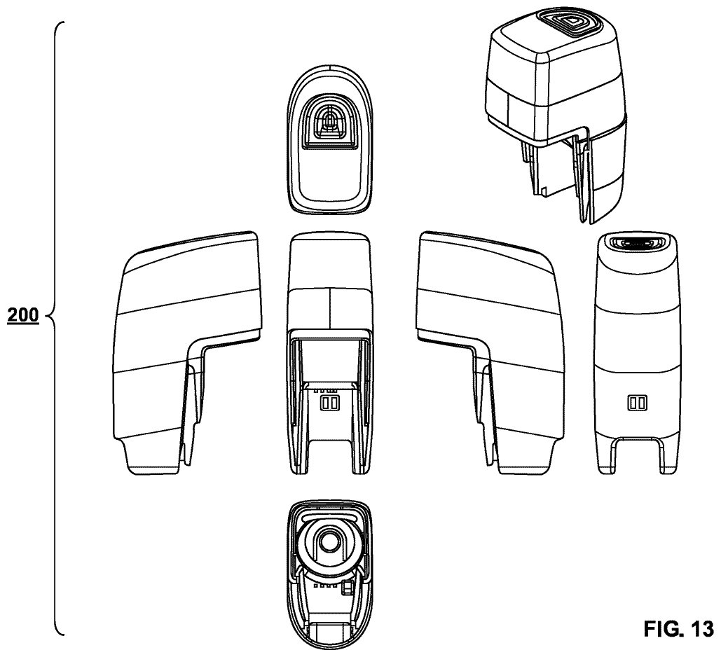

The six primary views and a perspective view of the drug cartridge 100, control cartridge 200, and device 1000 are illustrated in FIGS. 12, 13, and 14, respectively. These figures illustrate the various design views of the device and the individual components are not labeled for ease of viewing.

As utilized herein, the terms “comprises” and “comprising” are intended to be. construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

1. A modular handheld fluid dispensing system, the system comprising:
a fluid cartridge including an ampoule, a reservoir in fluid communication with the ampoule, an aperture designed to be selectively opened to allow fluid from the ampoule and accommodated in the reservoir to be dispensed therethrough, and an actuator designed to impart vibrations to the fluid cartridge to generate hydrodynamic pressure in the fluid to open the aperture and dispense the fluid;
a control cartridge being removably coupled to the fluid cartridge, the control cartridge including a power source and controller to initiate actuation of the actuator; and
a cover pivotally moveable between a closed configuration to prevent fluid from being dispensed through the aperture and an open configuration to permit the power source to be activated.

2. The system of claim 1, wherein the ampoule is threadedly received in the reservoir.

3. The system of claim 1, the system further including a vent tube designed to allow atmospheric pressure to enter the ampoule.

4. The system of claim 3, wherein a filter is arranged in the vent tube to prevent foreign contaminants from entering the ampoule.

5. The system of claim 1, wherein the fluid cartridge further includes a membrane arranged opposite the aperture.

6. The system of claim 5, wherein a pin portion of the membrane is received within the aperture to prevent fluid from exiting the aperture.

7. The system of claim 1, wherein the fluid cartridge further includes electrical contacts to electrically connect the fluid cartridge to the control cartridge.

8. The system of claim 7, wherein the electrical contacts include contacts to provide identification information about the ampoule to the control cartridge.

9. The system of claim 1, wherein the fluid cartridge further includes a latch designed to lock the fluid cartridge relative to the control cartridge.

10. The system of claim 1, wherein the controller includes a printed circuit board configured to send a control signal to the actuator to dispense the fluid, the printed circuit board is configured to:
control a number of doses that are ejected by the actuator; or
provide a signal to a user indicating a number of doses remaining in the ampoule.

11. The system of claim 1, wherein the cover is pivotally connected to the fluid cartridge such that the cover can pivot from a closed configuration covering the aperture to the open configuration.

12. The system of claim 11,
wherein the cover is designed to have a length extending from a pivot point to an edge, such that when the edge is placed against a cheek of a user, the system will be correctly located for dispensing the fluid, or
wherein the cover is mechanically connected to a switch arm and the switch arm is designed to activate the power source when the cover is in the open configuration and disengage the power source when the cover is in the closed configuration.

13. The system of claim 12, wherein the fluid cartridge includes a concave mirror having an orifice extending through a center of the concave mirror, the concave mirror being disposed proximate to and concentric with the aperture.

14. The system of any one of claim 1, wherein the ampoule contains lifitegrast ophthalmic solution.

15. A method of dispensing a fluid, the method comprising,
providing a liquid cartridge including an ampoule, a reservoir in fluid communication with the ampoule, an aperture designed to be selectively opened to allow the fluid to be dispensed therethrough, a cover, and an actuator designed to impart vibrations to the liquid cartridge to generate hydrodynamic pressure in the fluid to open the aperture and dispense the fluid;
receiving the liquid cartridge with a control cartridge, the control cartridge including a power source and a controller for sending a control signal to the actuator; and
pivoting the cover from a closed configuration, where the cover prevents fluid from being dispensed through the aperture, to an open configuration to activate the power source; and
activating the actuator to dispense the fluid with the controller.

16. The method of claim 15, further comprising,
opening the cover from the closed configuration to the open configuration; and
aligning the cover with an anatomical feature of a user to align the aperture with an eye of the user, or aligning a mirror, disposed about the aperture of the liquid cartridge, with the eye of the user to accurately dispense fluid to the eye.

17. The method of claim 15, further comprising,
removing the liquid cartridge from the control cartridge; and
inserting a new liquid cartridge into the control cartridge.

18. A method of assembling a device for dispensing fluid, the method comprising,
providing an ampoule containing a fluid to be dispensed and a liquid cartridge including a reservoir having an opening with a vent tube extending there through, an aperture designed to be selectively opened to allow the fluid to be dispensed therethrough, a cover, and an actuator designed to impart vibrations to the liquid cartridge to generate hydrodynamic pressure in the fluid to open the aperture and dispense the fluid
aligning the vent tube with a neck of the ampoule and connecting the ampoule to the liquid cartridge to form a combined ampoule and liquid cartridge; and
arranging the combined ampoule and liquid cartridge in a pouch.

19. The method of claim 18, wherein the combined ampoule and liquid cartridge are configured to be received within a control cartridge, the control cartridge including a power source and a controller for sending a control signal to the actuator.

20. The method of claim 18, wherein the ampoule contains lifitegrast ophthalmic solution.
